(19) United States
(12) Patent Application Publication
(10) Pub. No.: US 2020/0311781 A1
(43) Pub. Date: Oct. 1, 2020

(54) PROCESS AND SYSTEM FOR RATING A PHYSICIAN OFFICE

(71) Applicant: eSimplify Enterprises, Inc., Livonia, MI (US)

(72) Inventor: Pradeep Kalmat, Livonia, MI (US)

(73) Assignee: eSimplify Enterprises, Inc., Livonia, MI (US)

(21) Appl. No.: 16/834,294

(22) Filed: Mar. 30, 2020

Related U.S. Application Data
(60) Provisional application No. 62/825,354, filed on Mar. 28, 2019.

(51) Int. Cl. G06Q 30/02; G16H 10/60; G16H 40/20; G16H 70/20; G16H 50/70; G16H 15/00; G06Q 10/06; G06Q 10/10; G06Q 10/04

(52) U.S. Cl. CPC G06Q 30/0282; G16H 10/60; G16H 40/20; G16H 70/20; G06Q 10/04; G16H 15/00; G06Q 10/06398; G06Q 10/10; G06Q 30/0206; G16H 50/70

(57) ABSTRACT

A process for rating a physician office is provided. The process includes, within a computerized processor, operating programming to access data related to a patient panel of a doctor from a database, analyze the data to determine a list of patient diagnoses made by the doctor and corresponding treatments prescribed by the doctor, compare the corresponding treatments prescribed by the doctor to average treatments prescribed based upon the list of patient diagnoses, generate a report evaluating performance of the doctor based upon the comparing, and utilize the report to improve precision by correcting the corresponding treatments prescribed by the doctor to more closely correspond with average treatments prescribed.

### CROSS-REFERENCE TO RELATED APPLICATIONS

This application claims the benefit of U.S. Provisional Patent Application No. 62/825,354 filed on Mar. 28, 2019, the disclosure of which is hereby incorporated by reference.

### INTRODUCTION

The disclosure generally relates to a process and system for rating a physician office.

Physicians treat their clients and get reimbursed by government agencies and private insurers for treating a portion of their clients. In the United States, physicians treat their patients, submit billing data to the Centers for Medicare and Medicaid Services (CMS) and other private insurers, and get reimbursed by the government and Private Insurers for covered services. CMS and other government agencies publish data regarding these reimbursements. Such published data includes statistical information specific to doctors and doctors' offices, including but not limited to funds reimbursed, numbers of particular treatments or diagnosed codes provided for patients, number of patients seen, patient age demographics and population demographics.

### SUMMARY

A process for rating a physician office is provided. The process includes, within a computerized processor, operating programming to access data related to a patient panel of a doctor from a database, analyze the data to determine a list of patient diagnoses made by the doctor and corresponding treatments prescribed by the doctor, compare the corresponding treatments prescribed by the doctor to average treatments prescribed based upon the list of patient diagnoses, generate a report evaluating performance of the doctor based upon the comparing, and utilize the report to improve precision by correcting the corresponding treatments prescribed by the doctor to more closely correspond with average treatments prescribed.

In some embodiments, accessing data from the database includes accessing data from Centers for Medicare and Medicaid Services.

In some embodiments, generating the report includes alerting the doctor to risk of an audit by the Centers for Medicare and Medicaid Services.

In some embodiments, accessing data from the database includes accessing data from the American Medical Association.

In some embodiments, comparing the corresponding treatments prescribed by the doctor to the average treatments includes comparing the corresponding treatments prescribed by the doctor to standards published by the American Medical Association.

In some embodiments, accessing data from the database includes accessing data from a private insurance company.

In some embodiments, accessing data from the database includes accessing data from one of electronic medical records or electronic health records provided by the doctor.

In some embodiments, comparing the corresponding treatments prescribed by the doctor to the average treatments includes analyzing the data to determine a list of eligible treatments based upon the list of patient diagnoses made by the doctor and comparing the list of eligible treatments to the corresponding treatments prescribed by the doctor.

In some embodiments, generating the report includes estimating a lost revenue by the doctor based upon comparing the list of eligible treatments to the corresponding treatment prescribed by the doctor.

In some embodiments, the process further includes operating programming to determine a percentage of eligible treatments prescribed by the doctor based upon comparing the list of eligible treatments to the corresponding treatments prescribed by the doctor. In some embodiments, the process further includes, for a group of doctors of a same specialty as the doctor, operating programming to determine a list of eligible treatments for patients of the group of doctors based upon a list of patient diagnoses made by the group of doctors, determine corresponding treatments prescribed by the group of doctors based upon the list of patient diagnoses made by the group of doctors, and determine a percentage of eligible treatments prescribed by the group of doctors based upon comparing the list of eligible treatments for patients of the group of doctors to the corresponding treatments prescribed by the group of doctors. In some embodiments, comparing the corresponding treatments prescribed by the doctor to average treatments prescribed based upon the list of patient diagnoses includes comparing the percentage of eligible treatments prescribed by the doctor and the percentage of eligible treatments prescribed by the group of doctors.

In some embodiments, comparing the corresponding treatments prescribed by the doctor to the average treatments prescribed based upon the list of patient diagnoses includes determining a financial performance of the doctor as compared to an average doctor. In some embodiments, generating the report evaluating performance of the doctor includes reporting the financial performance of the doctor as compared to the average doctor.

According to one alternative embodiment, a process for rating a physician office is provided. The process includes, within a computerized processor, operating programming to access data related to a patient panel of a practice of doctors from a database, analyze the data to determine a list of patient diagnoses made by the practice of doctors and corresponding treatments prescribed by the practice of doctors, compare the corresponding treatments prescribed by the practice of doctors to average treatments prescribed based upon the list of patient diagnoses, generate a report evaluating performance of the practice of doctors based upon the comparing, and utilize the report to improve precision by correcting the corresponding treatments prescribed by the doctor to more closely correspond with average treatments prescribed.

In some embodiments, the process further includes operating programming to estimate a predicted caseload profile through a future timespan for the practice of doctors based upon the list of patient diagnoses made by the practice of doctors and corresponding treatments prescribed by the practice of doctors. In some embodiments, generating the report includes predicting whether the practice of doctors has sufficient resources to service the predicted caseload profile through the future timespan.

In some embodiments, comparing the corresponding treatments prescribed by the practice of doctors to the average treatments prescribed based upon the list of patient diagnoses includes comparing the corresponding treatments prescribed by the practice of doctors to data provided by Centers for Medicare and Medicaid Services.

In some embodiments, comparing the corresponding treatments prescribed by the practice of doctors to the average treatments prescribed based upon the list of patient diagnoses includes comparing the corresponding treatments prescribed by the practice of doctors to data provided by a state health system.

In some embodiments, comparing the corresponding treatments prescribed by the practice of doctors to the average treatments prescribed based upon the list of patient diagnoses includes comparing the corresponding treatments prescribed by the practice of doctors to American Medical Association data.

According to one alternative embodiment, a system for rating a physician office is provided. The system includes, within a computerized processor of a server device, operating programming to access data related to a patient panel of a doctor from a database, analyze the data to determine a list of patient diagnoses made by the doctor and corresponding treatments prescribed by the doctor, compare the corresponding treatments prescribed by the doctor to average treatments prescribed based upon the list of patient diagnoses, and generate a report evaluating performance of the doctor based upon the comparing.

In some embodiments, programming to access data from the database includes programming to access data from Centers for Medicare and Medicaid Services.

In some embodiments, programming to compare the corresponding treatments prescribed by the doctor to the average treatments includes programming to analyze the data to determine a list of eligible treatments based upon the list of patient diagnoses made by the doctor and compare the list of eligible treatments to the corresponding treatments prescribed by the doctor.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 illustrates a computerized server device configured to operate the methods disclosed herein, in accordance with the present disclosure.

FIG. 2 is a flowchart illustrating one exemplary embodiment of a process for rating a physician office, in accordance with the present disclosure.

FIG. 3 illustrates an alternative exemplary embodiment of a process for rating a physician office, in accordance with the present disclosure.

FIG. 4 illustrates an additional alternative exemplary embodiment of a process for rating a physician office, in accordance with the present disclosure.

FIG. 5 illustrates an additional alternative exemplary embodiment of a process for rating a physician office, in accordance with the present disclosure.

FIG. 6 illustrates an additional alternative exemplary embodiment of a process for rating a physician office, in accordance with the present disclosure.

### DETAILED DESCRIPTION

Physician's offices perform with varying levels of skill and efficiency. For a given population of patients, certain average medical conditions and proper diagnoses may be estimated. A given population may include any definition, for example, a group of patients with diabetes, a group of patients over 65 years of age, or a group of patients that have had a coronary bypass. Through analysis of data regarding operation of a doctor's office, if a given population sees a general practitioner, certain average diagnoses and treatments may be predicted (e.g, out of a thousand patients with a historical condition X, statistics show that Y % of them in a year will receive a certain diagnosis, with Z % with that diagnosis receiving a particular treatment.) If that given population sees a cardiologist, certain average diagnoses and treatments may be predicted. If that given population sees an orthopedic surgeon, certain average diagnoses and treatments may be predicted.

Analysis of data regarding operation of a doctor's office may include data gathered from a variety of sources. For example, data published by CMS may be analyzed. In another example, data published by private insurance companies may be analyzed. In another example, EMR/EHR from the files of a doctor's office or practice of doctors may be analyzed. Additionally alternatives include but are not limited to data provided by the American Medical Association (AMA), subgroups or specialty groups within the AMA, national health officials, state health officials, files from a hospital system, files from a corporation owning hospitals and facilities across multiple states, data provided by private interest groups, and data from other similar sources. The processes disclosed herein may be operated with any individual or any combination of the above or similar data sources.

Physicians treat patients, and as a result, submit billing data to CMS and other private insurers in order to be paid according to coverage under Medicare and Medicaid policy guidelines in addition to covered services as per private health insurers. The government publishes data for each physician, for example, including, for a particular time period, the doctor with specialty X treated 110 total patients, while treating 60 patients under Procedure code 111 for a total of $6,000 reimbursed, 30 patients under Procedure code 112 for a total of $4,500 reimbursed, and 20 patients under Procedure code 113 for a total of $8,000 reimbursed, for a total of $18,500 reimbursed. Averages for doctors practicing specialty X with a similar number of patients averaged 40 patients under diagnostic code 111 for a total of $4,000 reimbursed, 40 patients under diagnostic code 112 for a total of $6,000 reimbursed, and 30 patients under diagnostic code 113 for a total of $12,000 reimbursed, and additionally with additional treatments under other codes totaling $5,000, for a total of $27,000 reimbursed. Such results may be analyzed under a number of different schemes. According to a first exemplary analysis scheme, the above exemplary results may be analyzed to estimate a core competency rating for the physician. Assuming that the populations for doctors of a particular specialty tend to group around an average set of medical conditions, failures of a particular physician to diagnose according to industry averages, either in one sample or more preferably over a series of samples, may indicate that the doctor is missing diagnoses or is failing to use the most up to date industry practices. Further, missing codes or treatments may further indicate that a doctor is not up to date or is lagging behind in training or equipment. Such data may be processed to create a rating for the physician, providing a viewer with a way to judge the competency of the physician as compared to industry standards or standards within subsets of doctors.

Processes disclosed herein may compare a doctor's performance to industry standards. Industry standards may be established according to a number of embodiments. According to one embodiment, standards, practices, and data published by the AMA may be used to judge or evaluate the performance of a doctor or practice of doctors. Similarly, similar data from a specialty group within the AMA (e.g., the American Academy of Pediatrics, the American College of Cardiology, etc.) may be used to evaluate the performance of a doctor with a particular specialty or panel of patients with certain diagnoses. In another embodiment, data related to doctors in a particular city, state, or region may be used to evaluate the performance of a doctor within that geographical area.

According to a second exemplary analysis scheme, the above exemplary results may be analyzed to aid the physician's practice to be more profitable. For example, a company operating the analysis software may compare Medicare reimbursements for a physician or a practice including a plurality of physicians to average reimbursements for an average similar practice and identify differences between the practice and the average similar practice that could be used to make changes to make the practice more profitable. For example, in the above example, it may be demonstrated to the physician's practice that they are under-diagnosing certain codes as compared to average physician practices and that other practices servicing similar populations in the same specialty provide additional treatments and receive additional reimbursements under codes not utilized by the practice.

In another exemplary embodiment, Medicare data may be used to create intra-practice diagnostics, comparing physicians to each other. Such an application could be based upon the following elements: monitoring published Medicare data, filtering the Medicare data to determine practice specific metrics for each physician within a practice, comparing the metrics for the practice to red flag criteria, identifying outliers, and providing training recommendations, identifying lost opportunities, providing risk assessments.

In another exemplary embodiment, Medicare data may be used to create practice specific targeted marketing, for example, searching for practices that make less than a certain amount, practices with a specific threshold of a certain medical codes, practices with diagnosed inefficiencies. Such an application could be based upon the following elements: monitoring published Medicare data, filtering the Medicare data to determine practice specific metrics for a plurality of medical practices, and providing targeted marketing to one of the plurality of medical practices based upon the practice specific metrics.

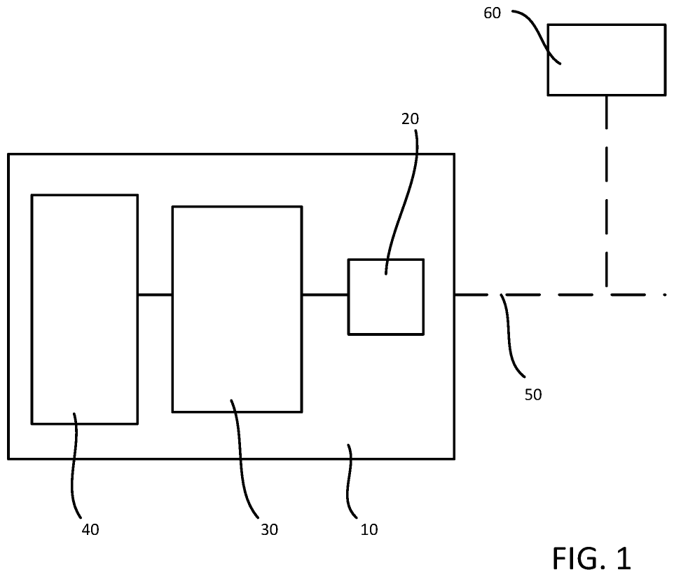

Referring now to the drawings, wherein the showings are for the purpose of illustrating certain exemplary embodiments and not for the purpose of limiting the same, FIG. 1 illustrates a computerized server device configured to operate the methods disclosed herein. Server device 10 is illustrated including communications device 20, computerized processor device 30, and durable memory storage device 40. Communications device 20 is illustrated configured to transmit and receive data over communications network 50. Communications device 20 is connected to and takes commands from computerized processor device 30.

Computerized processor device 30 includes a processor and random access memory (RAM) and is operable to execute computerized code or programming. Steps in the various exemplary processes and methods disclosed herein may be executed within one or more computerized modules operative within processor device 30, and may generally be used to monitor inputs, make automatic calculations or determinations, and provide an output based upon the calculations or determinations.

Durable memory storage device 40 includes a device capable of storing computerized or digital information and may include an exemplary hard drive, solid state drive, memory stick, or other similar known storage device.

Remote computerized device 60 is illustrated, such as a remote government server, which may provide information over communications network 50, such as published Medicare data.

Server device 10 is exemplary, a number of alternative configurations are envisioned, and the disclosure is not intended to be limited to the particular examples provided herein.

In some embodiments, the present methods and systems include additionally reviewing medical charts of the provider, not just the claims. Such medical records may include electronic medical records/electronic health records (EMR/EHR) and any other means an encounter is documented. This allows us to proactively review physicians thoroughness during the encounter. Claims don't typically detail and document the whole encounter. Also, sometimes physicians employ medical coders/billers that may or may not capture the whole encounter in the claims. By examining EMR/EHR documentation, the disclosed process may check and/or augment data acquired through claims analysis.

The present method and system may analyze primary and secondary diagnosis in the charts. Sometimes these are not reported in the claims. The present method and system may analyze family or personal history of patients, for example, taking racial or gender health trends or tendencies into account. The present method and system may consider “Does Medical treatment consider the various primary and secondary conditions in the episodal plan of care?”, “ Are medical decisions being made based on patient race, patient age, personal and family history?”, and “Do the claims data suggest or show the evidence of treatment based on family/personal history, age of patients and effectiveness of a plan of care considering the secondary conditions and possible risk of complication from conditions may not directly be present?” In some embodiments, the present system and method will track the patient encounters for twelve months post acute, emergency, or critical care in a hospital.

The present method and system may analyze just Medicare and similar government type data. In other embodiments, the present system and method may analyze both government published data and/or medical information from secondary sources such as electronic medical records to synthesize evaluations of doctors and practices for various comparative metrics as disclosed herein.

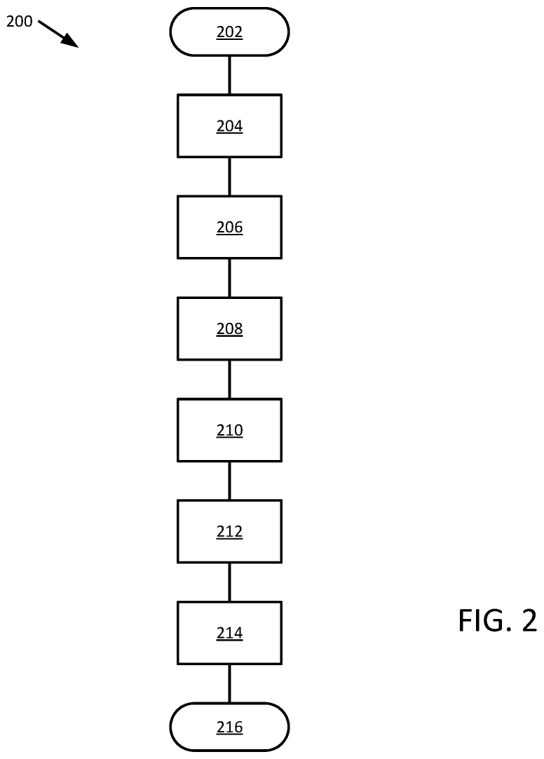

FIG. 2 is a flowchart illustrating one exemplary embodiment of a process for rating a physician office. A process 200 starts at step 202. At step 204, data is accessed or downloaded from a CMS database related to a particular doctor or a particular practice of doctors. At step 206, the data is examined, a patient panel or a list of patients served by the doctor or practice of doctors is analyzed, and a number of patients with a certain diagnosis eligible for a certain treatment or procedure is determined. At step 208, the data is examined, and a determination is made regarding how many times the doctor or practice of doctors actually prescribed or performed the certain procedure from step 206. At step 210, a percentage of eligible treatments or procedures actually prescribed or performed is generated. At step 212, a lost revenue value for the doctor or practice of doctors is estimated. At step 214, a report detailing values of steps 206, 208, 210, and 212 is generated. The report generated in step 214 may be utilized to diagnose lost opportunities for the doctor or practice of doctors in terms of generating revenue. Further, the report generated in step 214 may be utilized to improve a quality of care provided by the doctor or practice of doctors, improving the doctor or doctors' occurrence of treatment according to practices recommended by CMS and/or industry standards. The process 200 ends at step 216.

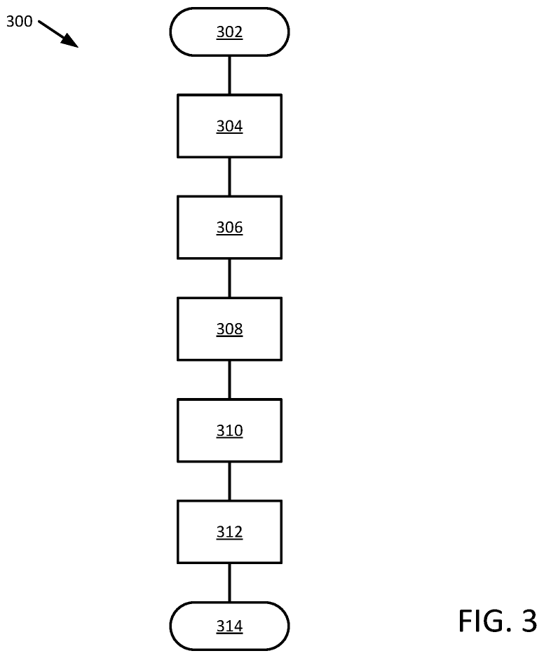

FIG. 3 illustrates an alternative exemplary embodiment of a process for rating a physician office. A process 300 starts at step 302. At step 304, data is accessed or downloaded from a CMS database related to a particular doctor or a particular practice of doctors. Additionally, data from EMR/EHR for the doctor or practice of doctors may be downloaded. At step 306, the data accessed in step 304 is analyzed, and, for each patient of the doctor or practice of doctors, a list of medical diagnoses is compiled. At step 308, a list of eligible treatments or procedures and eligible treatment dates for each patient is determined. At step 310, a schedule for sending out reminders to each of the patients related to the eligible treatments or procedures and the eligible treatment dates is generated. At step 312, reminders to schedule appointments are provided to each patient, for example, inviting the patient to connect with the doctor or practice's scheduling department. In another embodiment, at step 312, a report with the patient information, the eligible treatments or procedures, and the eligible treatment dates is generated and provided to a scheduling department for the doctor or practice of doctors. Such a process to provide for scheduling of appointments may further leverage available technology, such as calendar applications and invitations, text messaging, social media, and other similar technologies. At step 314, the process 300 ends.

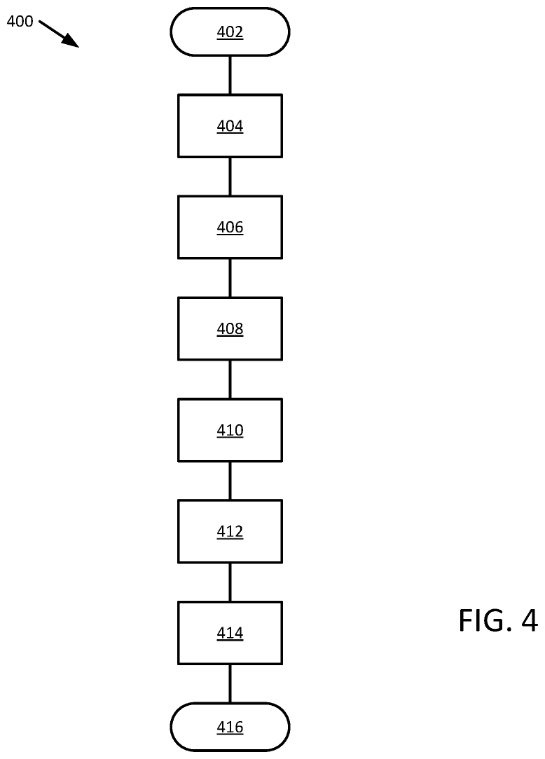

FIG. 4 illustrates an additional alternative exemplary embodiment of a process for rating a physician office. A process 400 starts at step 402. At step 404, data related to operation of a practice of doctors is access or downloaded. The data from step 404 may be accessed from CMS, EMR/EHR for the practice of doctors, or other similar sources. At step 406, based upon the data, a current caseload for the practice of doctors is determined. At step 408, based upon the data, a predicted caseload profile through a future timespan for the practice of doctors is estimated. The predicted caseload profile may take into account seasonal trends. The predicted caseload profile may take into account illness data such as an expected severity of a coming flu season. The predicted caseload may take into account changes in the local populace, such as local college semester schedules affecting patients visiting the practice of doctors. At step 410, a list of resources for the practice of doctors is input or determined. In one embodiment, the process may query the practice of doctors to input a list of doctors and medical professionals that will be available through the future timespan. In another embodiment, the process may utilize the accessed data to estimate resources available to the practice of doctors. At step 412, resources available to the practice of doctors through the future timespan is compared to the predicted caseload profile. At step 414, a report is generated predicting whether the practice of doctors has sufficient resources to service the predicted caseload profile through the future timespan. The report may be utilized to determine whether a new doctor needs to be hired, whether contract personnel need to be retained, and other similar information. At step 416, the process 400 ends.

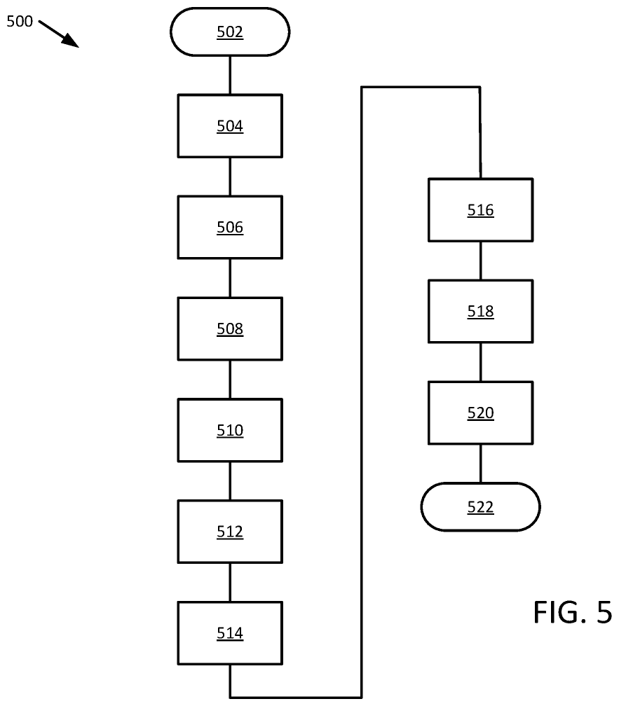

FIG. 5 illustrates an additional alternative exemplary embodiment of a process for rating a physician office. A process 500 starts at step 502. At step 504, data is accessed or downloaded from a CMS database related to a particular doctor. At step 506, based upon the accessed data, a list of eligible treatments or procedures for a patient panel of the doctor is determined. At step 508, based upon the accessed data, a determination is made regarding how many of the eligible treatments or procedures the doctor actually prescribed or performed. At step 510, a determination is made regarding a percentage of each eligible procedure that the doctor actually prescribed or performed. At step 512, data is accessed or downloaded from the CMS database related to a grouping of doctors, and for the grouping of doctors, a list of eligible procedures for a patient panel of the grouping of doctors is determined. At step 514, based upon the data for the grouping of doctors, a determination is made regarding how many of the eligible procedures the grouping of doctors actually prescribed or performed. At step 516, a determination is made regarding a percentage of each eligible procedure that the grouping of doctors actually prescribed or performed. At step 518, a comparison is made between what percentage of each eligible procedure the doctor performed versus what percentage of each eligible procedure the grouping of doctors actually prescribed or performed. At step 520, a report describing the comparison of step 518 is generated. This report may be used to improve the financial performance of the doctor. This report may be used to assist the doctor in prescribing the most up to date treatments for particular conditions and diagnoses. This report may be used to identify outlier performance of the doctor, for example, over prescription of a certain treatment in relation to other doctors in the industry. The grouping of doctors may be defined to include a plurality of doctors in the same practice as the doctor, a practice of doctors practicing locally to the doctor, doctors in a particular state or region, doctors employed by a particular company or hospital system, or doctors within a nation. At step 522, the process 500 ends.

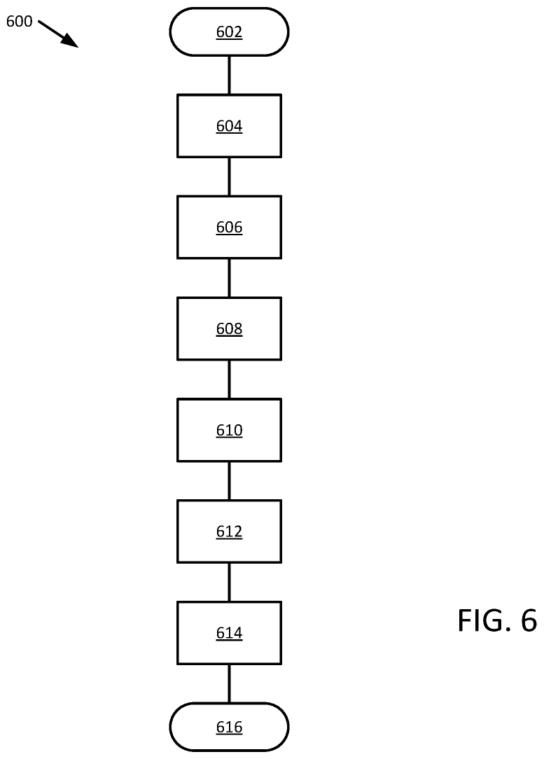

FIG. 6 illustrates an additional alternative exemplary embodiment of a process for rating a physician office. A process 600 starts at step 602. At step 604, data is accessed or downloaded from a CMS database related to a particular doctor. At step 606, based upon the accessed data, a patient panel profile is determined. The patient panel profile may include diagnosed conditions and a list of eligible treatments. At step 608, a determination is made regarding treatments that the doctor performed. At step 610, data is accessed or downloaded from the CMS database related to an average patient panel profile for and treatments provided by average doctors similar to the particular doctor. At step 612, the treatments performed by the doctor are compared to the treatments performed by the average doctors. At step 614, alerts may be generated based upon the comparison. Alerts may include alerting the doctor that she or he is over or under performing certain treatments. Alerts may include alerting the doctor that she or he is losing financial opportunities by under performing certain treatments. Alerts may include alerting the doctor that certain treatment value may cause the CMS auditors to flag the doctor for an audit. At step 616, the process 600 ends.

Processes 200, 300, 400, 500, and 600 are provided as examples of the disclosed process, utilizing data regarding treatments provided by doctors to rate and/or improve performance of a doctor's office or a practice of doctors. A number of variations to processes 200, 300, 400, 500, and 600 are envisioned, and the disclosure is not intended to be limited to the examples provided herein.

Operation of a doctors office or a practice of doctors may include a single doctor, a plurality of doctors, doctors and staff, such as one or physician assistants and/or registered nurses and/or social workers and/or similar personnel that may be responsible for treatment of patients. A practice of doctors may include an entire hospital or hospital system including multiple facilities. Data, analyses, and reports described herein may include data for a single doctor, a single classification of doctors in a practice, a group or doctors, medical practitioners within a certain practice, or medical practitioners within a certain classification or geographical area. A wide variety of filters and classifications may be used to analyze data available from CMS and other sources, and the disclosure is not intended to be limited to the particular examples provided herein.

While the best modes for carrying out the disclosure have been described in detail, those familiar with the art to which this disclosure relates will recognize various alternative designs and embodiments for practicing the disclosure within the scope of the appended claims.

What is claimed is:

1. A process for rating a physician office, comprising:
within a computerized processor, operating programming to:
access data related to a patient panel of a doctor from a database;
analyze the data to determine a list of patient diagnoses made by the doctor and corresponding treatments prescribed by the doctor;
compare the corresponding treatments prescribed by the doctor to average treatments prescribed based upon the list of patient diagnoses;
generate a report evaluating performance of the doctor based upon the comparing; and
utilize the report to improve precision by correcting the corresponding treatments prescribed by the doctor to more closely correspond with average treatments prescribed.

2. The process of claim 1, wherein accessing the data from the database includes accessing data from a Centers for Medicare and Medicaid Services.

3. The process of claim 2, wherein generating the report includes alerting the doctor to risk of an audit by the Centers for Medicare and Medicaid Services.

4. The process of claim 2, wherein accessing the data from the database includes accessing data from an American Medical Association.

5. The process of claim 4, wherein comparing the corresponding treatments prescribed by the doctor to the average treatments includes comparing the corresponding treatments prescribed by the doctor to standards published by the American Medical Association.

6. The process of claim 2, wherein accessing the data from the database includes accessing data from a private insurance company.

7. The process of claim 2, wherein accessing the data from the database includes accessing data from one of electronic medical records or electronic health records provided by the doctor.

8. The process of claim 1, wherein comparing the corresponding treatments prescribed by the doctor to the average treatments includes:
analyzing the data to determine a list of eligible treatments based upon the list of patient diagnoses made by the doctor; and
comparing the list of the eligible treatments to the corresponding treatments prescribed by the doctor.

9. The process of claim 8, wherein generating the report includes estimating a lost revenue by the doctor based upon comparing the list of eligible treatments to the corresponding treatments prescribed by the doctor.

10. The process of claim 8, further comprising operating programming to:
determine a percentage of eligible treatments prescribed by the doctor based upon comparing the list of the eligible treatments to the corresponding treatments prescribed by the doctor;
for a group of doctors of a same specialty as the doctor, determine a list of eligible treatments for patients of the group of doctors based upon a list of patient diagnoses made by the group of doctors;
determine corresponding treatments prescribed by the group of doctors based upon the list of patient diagnoses made by the group of doctors; and
determine a percentage of eligible treatments prescribed by the group of doctors based upon comparing the list of the eligible treatments for patients of the group of doctors to the corresponding treatments prescribed by the group of doctors; and
wherein comparing the corresponding treatments prescribed by the doctor to the average treatments prescribed based upon the list of the patient diagnoses includes comparing the percentage of eligible treatments prescribed by the doctor and the percentage of eligible treatments prescribed by the group of doctors.

11. The process of claim 1, wherein comparing the corresponding treatments prescribed by the doctor to the average treatments prescribed based upon the list of patient diagnoses includes determining a financial performance of the doctor as compared to an average doctor; and
wherein generating the report evaluating the performance of the doctor includes reporting the financial performance of the doctor as compared to the average doctor.

12. A process for rating a physician office, comprising:
within a computerized processor, operating programming to:
access data related to a patient panel of a practice of doctors from a database;
analyze the data to determine a list of patient diagnoses made by the practice of doctors and corresponding treatments prescribed by the practice of doctors;
compare the corresponding treatments prescribed by the practice of doctors to average treatments prescribed based upon the list of patient diagnoses;
generate a report evaluating performance of the practice of doctors based upon the comparing; and
utilize the report to improve precision by correcting the corresponding treatments prescribed by the doctor to more closely correspond with average treatments prescribed.

13. The process of claim 12, further comprising operating programming to estimate a predicted caseload profile through a future timespan for the practice of doctors based upon the list of patient diagnoses made by the practice of doctors and the corresponding treatments prescribed by the practice of doctors; and
wherein generating the report includes predicting whether the practice of doctors has sufficient resources to service the predicted caseload profile through the future timespan.

14. The process of claim 12, wherein comparing the corresponding treatments prescribed by the practice of doctors to the average treatments prescribed based upon the list of patient diagnoses includes comparing the corresponding treatments prescribed by the practice of doctors to data provided by a Centers for Medicare and Medicaid Services.

15. The process of claim 12, wherein comparing the corresponding treatments prescribed by the practice of doctors to the average treatments prescribed based upon the list of patient diagnoses includes comparing the corresponding treatments prescribed by the practice of doctors to data provided by a state health system.

16. The process of claim 12, wherein comparing the corresponding treatments prescribed by the practice of doctors to the average treatments prescribed based upon the list of patient diagnoses includes comparing the corresponding treatments prescribed by the practice of doctors to American Medical Association data.

17. A system for rating a physician office, comprising:
within a computerized processor of a server device, operating programming to:
access data related to a patient panel of a doctor from a database;
analyze the data to determine a list of patient diagnoses made by the doctor and corresponding treatments prescribed by the doctor;
compare the corresponding treatments prescribed by the doctor to average treatments prescribed based upon the list of patient diagnoses; and
generate a report evaluating performance of the doctor based upon the comparing.

18. The system of claim 17, wherein programming to access the data from the database includes programming to access data from a Centers for Medicare and Medicaid Services.

19. The system of claim 17, wherein programming to compare the corresponding treatments prescribed by the doctor to the average treatments includes programming to:
analyze the data to determine a list of eligible treatments based upon the list of patient diagnoses made by the doctor; and
compare the list of eligible treatments to the corresponding treatments prescribed by the doctor.
